(19) United States
(12) Patent Application Publication
(10) Pub. No.: US 2025/0345539 A1
(43) Pub. Date: Nov. 13, 2025

(54) JET VENTILATION APPARATUS AND METHOD

(71) Applicant: Pipeline Medical Products LLC, Weymouth, MA (US)

(72) Inventors: John Morgan, Weymouth, MA (US); Paul Imaoka, Jacksonsville, FL (US); Brandon Verwijst, Palatine, IL (US)

(21) Appl. No.: 19/203,591

(22) Filed: May 9, 2025

Related U.S. Application Data
(60) Provisional application No. 63/645,416, filed on May 10, 2024.

(51) Int. Cl. A61M 16/00; A61M 16/04; A61M 16/10

(52) U.S. Cl. CPC A61M 16/0096; A61M 16/0486; A61M 16/1005; A61M 2205/3344; A61M 2207/00; A61M 2230/43

(57) ABSTRACT

A jet ventilation assembly that has a conduit having a luer fitting on a proximal end and configured to fluidly couple the luer fitting to a distal end of the conduit and a centering assembly coupled to the conduit at the distal end. The centering assembly has a hub portion that is movable along the conduit.

### CROSS REFERENCE TO RELATED DISCLOSURE

The present disclosure claims the benefit of U.S. Provisional Application No. 63/645,416 filed 10 May 2024, the contents of which being incorporated herein in entirety by reference.

### FIELD OF THE DISCLOSURE

The present disclosure generally relates to a jet ventilation tube and more specifically to a jet ventilation tube having improved centering and monitoring capabilities.

### BACKGROUND

Jet ventilation tubes minimize obstruction while providing fluid flow to a patient. Conventional jet ventilation tubes are difficult to monitor and inconsistently place a tube outlet among other things.

During tracheal, laryngeal and lung surgery it is important to keep the patient properly oxygenated and ventilated. Traditionally, this is accomplished with a standard large diameter endotracheal tube which is used to control the flow of fluids to the patient's lungs. These are utilized due to the large volume of gas that is needed to oxygenate and ventilate the patient. In many airway and lung procedures these larger tubes create technical challenges for the surgeon. The larger diameter endotracheal tubes can limit the surgeons access to the airway or lungs and inhibit the ability to perform the procedure optimally. By taking up much of the operative field, they can limit the ability to use surgical instruments or devices efficiently. If employed, these large diameter tubes can also potentially damage the surrounding tissue during advancement or while being manipulated to attain better access during surgery.

A small diameter subglottic jet ventilation catheter with high pressure jet ventilation will allow for the appropriate oxygenation and ventilation and at the same time provide optimal unobstructed surgical access. Previous subglottic jet ventilation catheters have only offered a single monitor lumen. This made measuring both tracheal pressure and EtCO2 concurrently challenging intraoperatively. Additionally, when the distal opening of the monitoring lumen is exposed and unprotected, it is susceptible to becoming obstructed with tissue or fluid when coming in contact with the patient's anatomy during or after placement, which compromises or terminates the ability to monitor gases intraoperatively.

The fluid introduced through the endotracheal tube is often gaseous and includes oxygen to ensure that the patient is appropriately oxygenated and ventilated during the procedure. Due to the small diameter of some subglottic jet ventilation catheters the fluid exits the catheter with a greater force compared to other known methods and attention must be given to this increased force. The high-pressure jet stream of fluid that exits the catheter may cause harm to the patient if it is directly in contact with the tracheal wall or other tissue. In addition, the conventional small diameter catheters instability in the tracheal lumen creates is a potential for it to whip around or move significantly as the fluid is discharged. Additionally, fluid exiting a jet catheter at high pressure against tissue or the tracheal wall could potentially cause turbulent air flow to the lungs, among other things.

### SUMMARY

The present disclosure may comprise one or more of the following features and combinations thereof.

The first embodiment is the small diameter flexible conduit which is made of a unique single extrusion, triple lumen PTFE design which allows for fluid delivery through the main jet lumen and in addition there are two independent monitoring lumen.

The second embodiment is that both independent monitor ports distal openings are protected by and encased by the centering assembly in the monitoring zone, thus limiting direct tissue and tracheal wall contact, such direct contact could possibly lead to the occlusion of the distal opening of the monitoring lumen and hinder active monitoring. The monitoring zone protects the lumen openings and allows for monitoring pressures and gases within the trachea in the area surrounding the monitoring zone.

The third embodiment is the aforementioned centering assembly, which has a free-floating design. The proximal end of the centering assembly may be coupled to the single extrusion, three lumen conduit. Whereas the distal aspect of the centering assembly is free-floating and movable relative to the conduit (i.e., it freely moves over the distal aspect of a single lumen extrusion of the conduit). The conduit defines a lumen that communicates with the main jet lumen which originates at the proximal end of the conduit at the luer lock. This is where the fluid is delivered to the patient during the procedure.

Fourth, is the unique “flex tip” of the “centering assembly”. The distal end of the “centering assembly” floats freely over the distal aspect of the main jet lumen of the conduit. This results in a shock absorber like action. When the jet catheter is advanced into the anatomy and the “flex tip” is engaged, the “centering assembly” will slide proximally over the distal conduit until it hits the stop on the main conduit, which will firm up the flanges to deflect the “centering assembly” off of the anatomy or point of resistance. Once past the bend in the flanges or on extraction the reverse happens. The flex tip will disengage from the stop on the conduit and free float distally again on the conduit and become more pliable and conform better to the native anatomy and it also has the ability to flatten due to the “flex tip”.

This fifth embodiment speak directly to the engineering and interaction of the “flex flange” and the “flex tip”. The “flex flange” is unique in the it works and harmony with the action of the “flex tip” and allows for the centering assembly to either be firmly at it resting shape and size when the “flex tip” is engaged and allows for it to be collapsible when the “flex tip” is disengaged. The “flex flange” is unique in this ability.

The sixth embodiment is the addition of a second independent monitoring port originating at the longer proximal luer fitting and ending at the distal opening within the “monitoring zone”.

The seventh embodiment is that the centering assembly allows for the catheter assembly to become biased towards the center of the trachea once in the desired position in the trachea. Then the fluids that is delivered through the conduit will exit the most distal aspect of the conduit in a laminar flow fashion. This allows for non-turbulent, laminar flow of the fluids into the patient's lungs.

The eighth embodiment are the proximal markers on the conduit exterior which will allow the surgeon to know the exact distance from the most distal aspect of the conduit where the fluid exits to the lungs in a laminar fashion. This will allow for more precise placement of the jet tube and better able to adapt placement to individual anatomy during procedures.

The unique design of this disclosure has several prominent advantages over existing devices. Among other things, the present disclosure has independent dual monitoring lumen built into our single, triple lumen conduit and are protected within the “monitoring zone” inside the centering assemble. In addition, the centering assembly presented herein has a unique flex tip design that is possible due to the engineered interface of the distal conduit and the centering assembly. The flex tip engages or disengages the flanges of the centering assembly.

One embodiment is a jet ventilation assembly that has a conduit having a luer fitting on a proximal end and configured to fluidly couple the luer fitting to a distal end of the conduit and a centering assembly coupled to the conduit at the distal end. The centering assembly has a flex tip that is movable along the conduit.

In one example of this embodiment, the conduit defines a first lumen and a second lumen therein, the first lumen configured to provide oxygen to a patient and the second lumen configured to provide monitoring information to a user. In part of this example, the conduit defines a third lumen, the third lumen configured to provide additional monitoring information to a user. In another part of this example, the first lumen terminates at a location proximate the flex tip and is configured to direct fluid through the first lumen out the distal end of the flex tip. In one aspect of this part, the second lumen terminates at a monitoring zone of the centering assembly.

In another example of this embodiment, the centering assembly has at least one flex flange fixedly coupled to the conduit on one end and coupled to the flex tip on the opposite end. In yet another example, the centering assembly comprises a plurality of flex flanges each fixedly coupled to the conduit on one end and coupled to the flex tip on the opposite end.

In one example, the first lumen terminates at a location proximate the flex tip and the second and third lumen terminate at a monitoring zone of the centering assembly. Y et another example includes a second luer fitting configured to fluidly couple the second lumen to a first medical device and a third luer fitting configured to couple the third lumen to a second medical device. In part of this example, the second lumen is configured to fluidly couple the first medical device to the monitoring zone to measure a pressure in a patent's trachea. In another part of this example, the third lumen is configured to fluidly couple the second medical device to the monitoring zone to monitor CO2 levels.

In another example of this embodiment, the conduit is formed of PTFE. In yet another example, the centering assembly is formed of PEBAX. However, other materials for both the conduit and the centering assembly are considered herein.

In another example, the centering assembly can elastically deflect to allow the flex tip to move along the conduit to absorb shock during insertion. In yet another example, the conduit defines a stop configured to contact the flex tip when the centering assembly is moved to a maximum deflection.

Yet another embodiment of this disclosure is a method of manufacturing a jet ventilation assembly. The method includes injecting a material into a mold that defines a centering assembly having a flex tip and a plurality of flex flanges, wherein each of the flex flanges are formed extending from the flex tip, obtaining a tri-lumen conduit, and coupling the centering assembly to the tri-lumen conduit by heat-welding the plurality of flex flanges to one another around the tri-lumen conduit.

In one example of this embodiment, the flex tip is formed with a through hole and at least one lumen of the tri-lumen conduit is positioned at least partially in the through hole. Another example of this embodiment includes applying a primer and an adhesive to the flex flanges and the tri-lumen conduit to further couple the centering assembly to the tri-lumen conduit. In one part of this example, the adhesive is a cyanoacrylate.

In another example of this embodiment, the centering assembly is formed of PEBAX, and the tri-lumen conduit is formed of PTFE.

These and other features of the present disclosure will become more apparent from the following description of the illustrative implementations.

### BRIEF DESCRIPTION OF THE DRAWINGS

The above-mentioned aspects of the present disclosure and the manner of obtaining them will become more apparent and the disclosure itself will be better understood by reference to the following description of the implementations of the disclosure, taken in conjunction with the accompanying drawings, wherein:

FIG. 1 is an elevated perspective view of the jet ventilation tube;

FIG. 2 is a side view of the jet ventilation tube of FIG. 1;

FIG. 3 is a front view of the jet ventilation tube of FIG. 1;

FIG. 4 is a back view of the jet ventilation tube of FIG. 1;

FIG. 5 is a detailed view of a centering assembly of the jet ventilation tube of FIG. 1;

FIG. 6 is a section front view of the centering assembly of the jet ventilation tube of FIG. 1;

FIG. 7 is a section side view of the centering assembly of the jet ventilation tube of FIG. 1;

FIG. 8 is an exemplary flowchart illustrating the use of the jet ventilation tube of FIG. 1;

FIG. 9a is an exploded partial view of the jet ventilation tube of FIG. 1;

FIG. 9b is a partial view of the jet ventilation tube of FIG. 1 with the centering assembly removed;

FIG. 10a is a detailed view of the centering assembly in a neutral orientation;

FIG. 10b is a detailed view of the centering assembly in an engaged orientation; and

FIG. 10c is a detailed view of the centering assembly in a collapsed orientation.

Corresponding reference numerals are used to indicate corresponding parts throughout the several views.

### DETAILED DESCRIPTION

The implementations of the present disclosure described below are not intended to be exhaustive or to limit the disclosure to the precise forms in the following detailed description. Rather, the implementations are chosen and described so that others skilled in the art may appreciate and understand the principles and practices of the present disclosure.

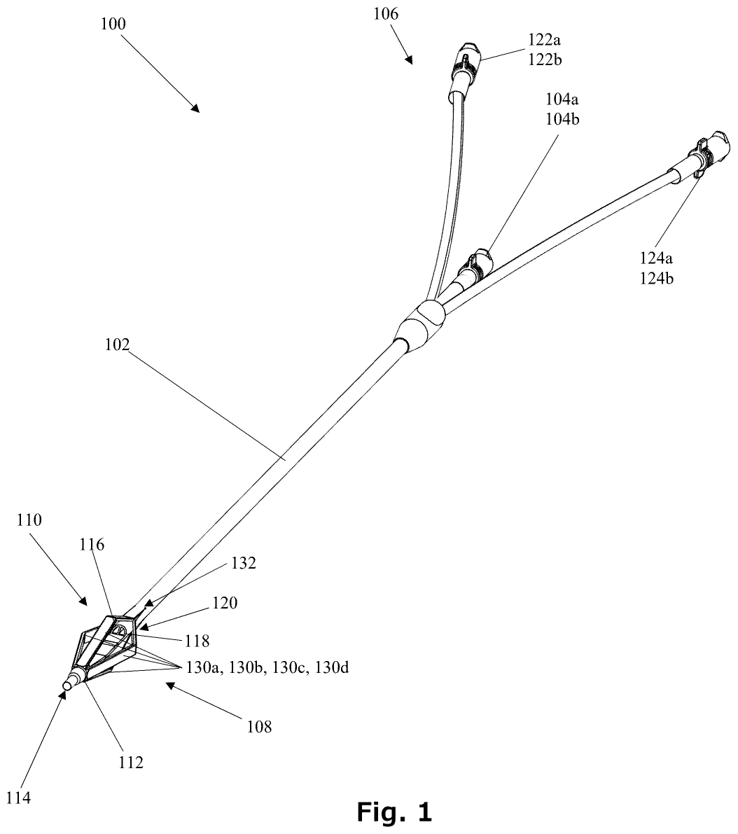

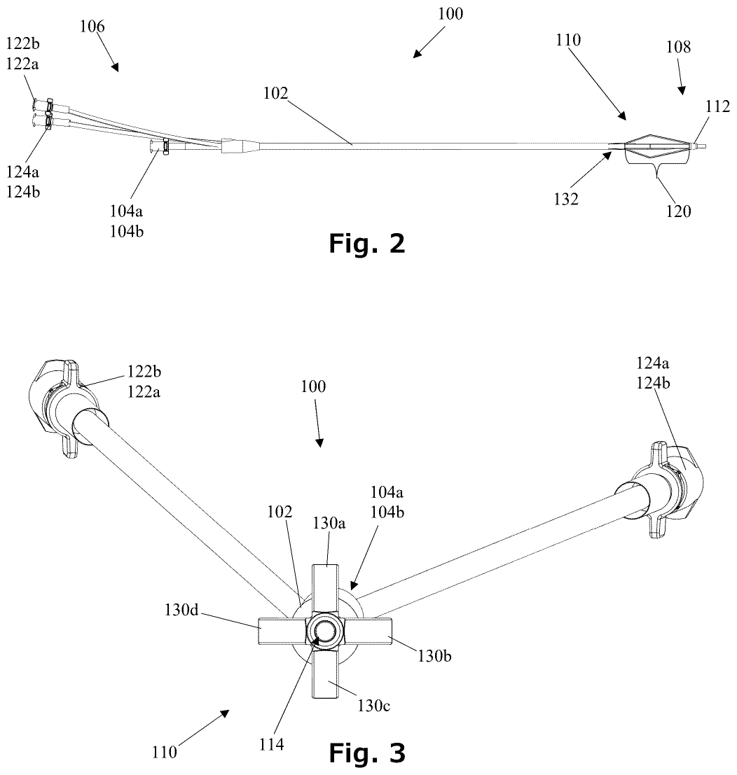

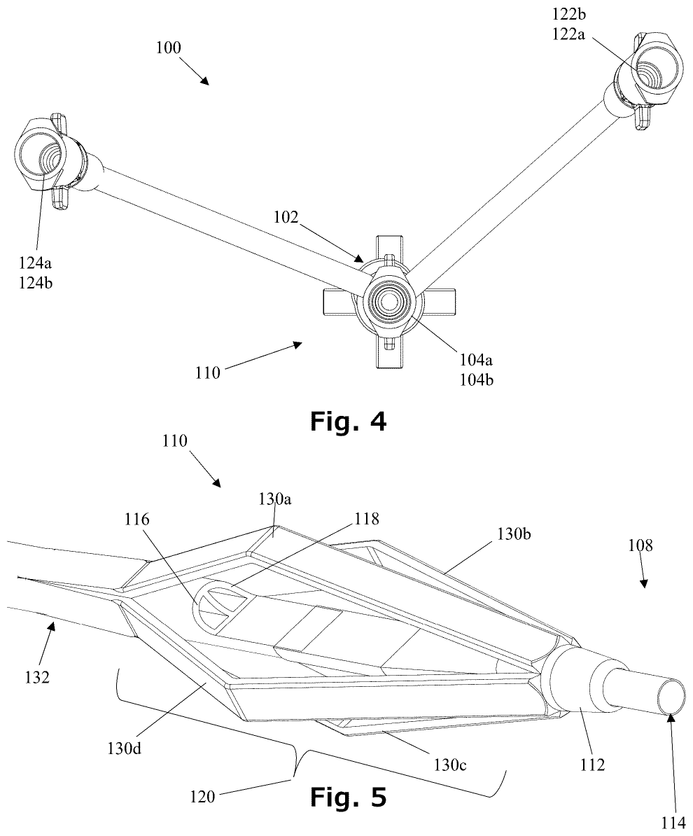

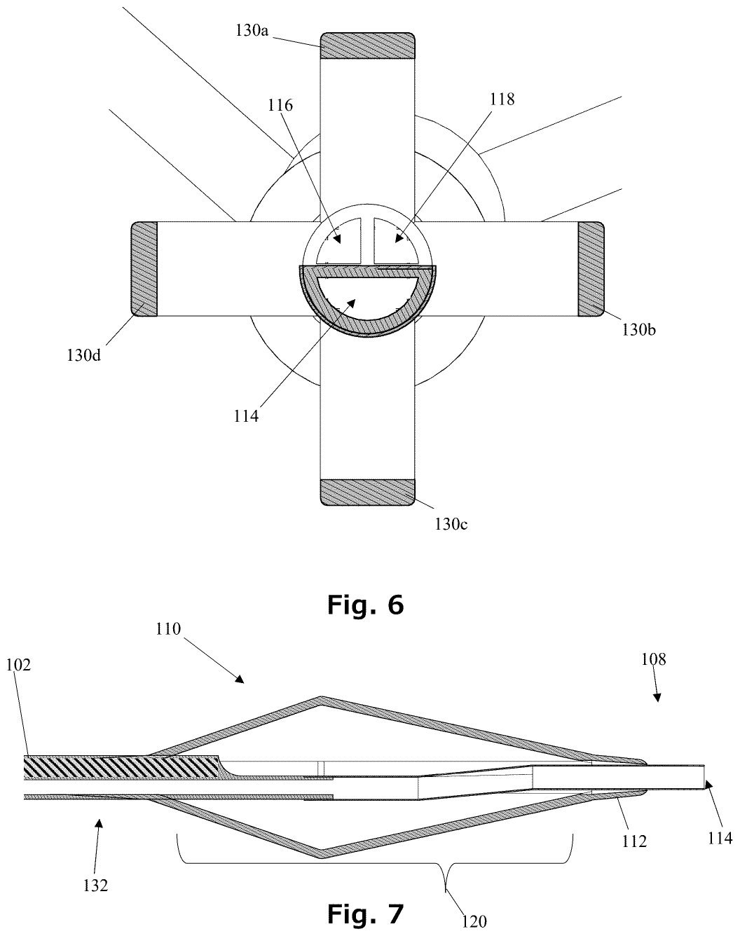

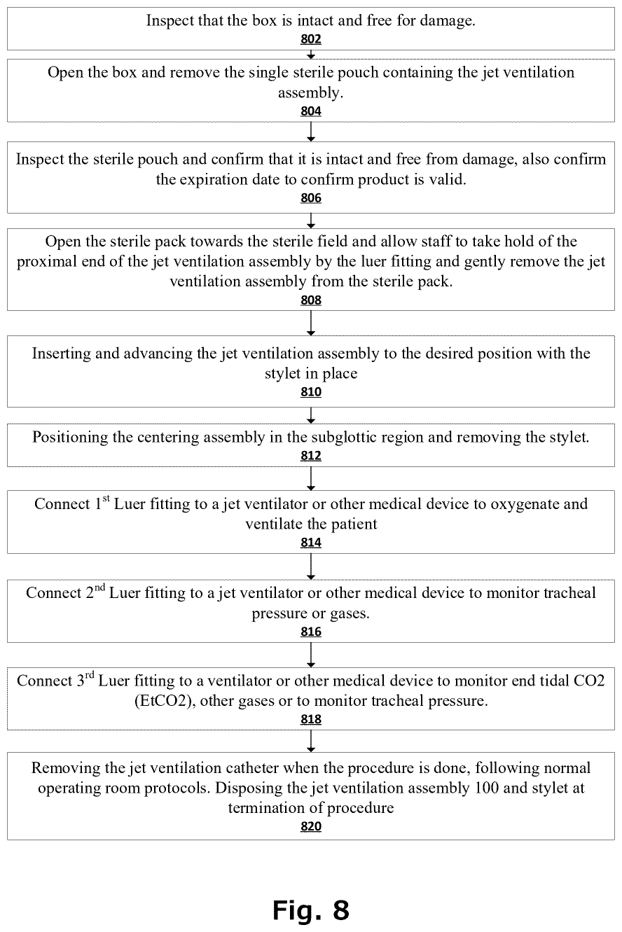

Referring now to FIG. 1 is a jet ventilation assembly 100 is illustrated shown isolated from a ventilator or other medical device. The jet ventilation assembly 100 which may be formed, in part, from an extruded PTFE tri-lumen conduit 102. The jet ventilation assembly 100 has a proximal end 106 and a distal end 108. The conduit 102 has its proximal opening 104a on the proximal end 106 and the distal or first opening at a distal terminus of a first lumen 114 at the distal end 108. The proximal opening 104a may have a luer fitting 104b. The luer fitting 104b may provide a location to fluidly couple to the proximal opening 104a of conduit 102 to a ventilator or other medical device to selectively provide fluid through the conduit 102 and out of the opening of the first lumen 114 at the distal end 108.

Also depicted in FIG. 1 is the 2^{nd} luer fitting 122b at a 2^{nd} proximal opening 122a and the 3^{rd} luer fitting 124b at a 3^{rd} proximal opening 124a on the proximal end 106. The proximal end 106 of the conduit 102 has proximal openings at the 2^{nd} luer fitting 122b and the 3^{rd} luer lock fitting 124b, which communicate directly with the correspondingly numbered 2^{nd} lumen 116 and 3^{rd} lumen 118 which open in a “monitoring zone” 120 of the centering assembly 110. The 2^{nd} luer fitting 122b and 3^{rd} luer fitting 124b may each provide independent connections to a ventilator or other medical device to selectively provide fluid through the conduit and also be used for the monitoring of gases and pressure within the trachea when connected to a ventilator or other medical device.

A base end 132 of the centering assembly 110 is coupled to the conduit 102 at a location biased towards the distal end 108 of the conduit 102. The 2^{nd} lumen 116 and the 3^{rd} lumen 118 open in the monitoring zone 120 which is protected on 4 sides by flex flanges 130a, 130b, 130c, 130d of the centering assembly 110. The monitoring zone 120 is the interior area in the centering assembly 110 where gases and pressures are monitored when either the 2^{nd} luer fitting 122b or the 3^{rd} luer fitting 124b are fluidly coupled to a ventilator or other medical device. The main jet line originates at opening 104a of the conduit 102 and communicates with the first lumen 114.

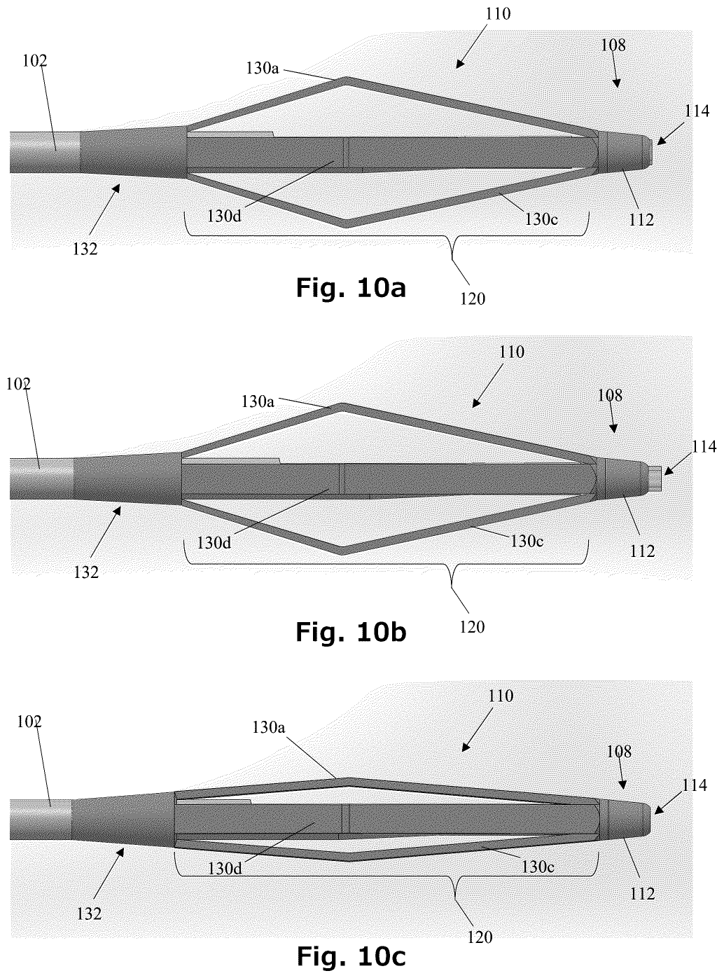

The 1^{st} lumen 114 extends past the the 2^{nd} lumen 116 and the 3^{rd} lumen 118, which terminate in the monitoring zone 120, and the first lumen 114 extends into or through part of a flex tip 112 of the centering assembly 110. The flex tip 112 allows for the main jet or 1^{st} lumen 114 in the conduit 102 to float freely to the most distal aspect of the centering assembly 110. This works in conjunction with the flex flanges 130a, 130b, 130c, 130d on the centering assembly 110 to allow a shock absorber like action. When the flex tip 112 is engaged (pushed toward proximal end 106) the flex flanges 130a, 130b, 130c, 130d on the centering assembly 110 respond by expanding relative to a neutral orientation (see FIG. 10a) of the centering assembly 110 be elastically deforming from a neutral position. This allows for the flex tip 114 and flex flanges 130a, 130b, 130c, 130d to deflect the centering assembly 110 and direct the first lumen 114 towards a center portion of the patient when in use. Conversely when the flex tip 114 is moved toward the distal end 108 the centering assembly 110 becomes oriented in a collapsed orientation (see FIG. 10c) that may orient the flex flanges 130a, 130b, 130c, 130d to be near completely flat against the first lumen 114.

The conduit 102 can be made of Polytetrafluoroethylene (“PTFE”) or any other material that can provide sterile, fluidly sealed inner channel. In one example, the tri-lumen conduit 102 may be formed via an extrusion process. However, any known method for forming a tri-lumen conduit is considered herein.

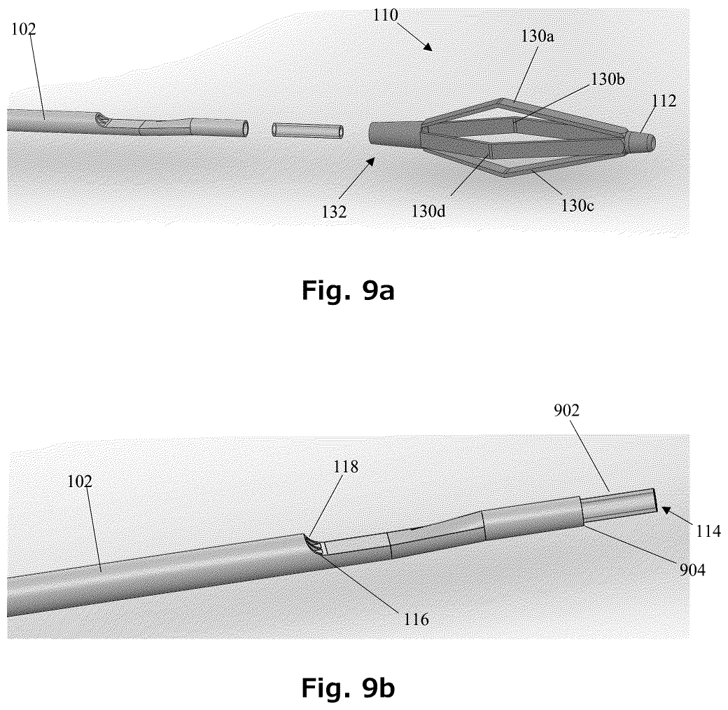

Referring now to FIGS. 9a and 9b, an exploded view (FIG. 9a) and a partial view with the centering assembly 110 removed (FIG. 9b) are illustrated. In one example of this disclosure, the distal section of the first lumen 114 may be coupled to a single lumen insertion 902. The single lumen insertion 902 may be sized to allow the flex tip 112 to slide thereover while the first lumen 114 may have a relatively larger diameter at the coupling point to form a stop 904 for the flex tip 112. In this configuration, the flex tip 112 may be configured to slide along the single lumen insertion 902 to allow the centering assembly 110 to deflect upon contact with tissue during insertion. However, the stop 904 may contact the flex tip 112 when the centering assembly 110 is move to a maximum deflection (see FIG. 10b) to prevent the centering assembly 110 from deforming greater than desired.

Referring now to FIG. 10a, the centering assembly 110 is illustrated in a neutral orientation wherein there is not a significant force acting on the centering assembly 110. In the neutral orientation, the centering assembly 110 is fixed on the proximal end to the conduit 102 and slidable relative to the conduit 102 along the single lumen insertion 902 if a force is exerted onto the centering assembly 110 in either direction. While the single lumen insertion 902 is illustrated extending past the flex tip 112 in FIG. 10a, in other embodiments the single lumen insertion 902 may terminate within the flex tip 112 when in the neutral orientation.

FIG. 10b illustrates the centering assembly 110 in an engage orientation wherein the flex tip 112 is moved along the single lumen insertion 902 to contact the stop 904. In this example, when a force is applied on the distal end of the centering assembly 110 in the direction of the proximal end, as may be typical during insertion, the centering assembly 110 moves along the single lumen insertion 902 until it contacts the stop 904 where the single lumen insertion 902 is coupled to the first lumen 114. During this action, the flex flanges 130a, 130b, 130c, 130d deflect outwardly away from the first lumen 114 to provide deflection off any adjacent wall. In use, this allows the centering assembly 110 to bias the first lumen 114 towards the center of an airway when the flex tip 112 contacts an obstruction because the flex flanges 130a, 130b, 130c, 130d expand as the flex tip 112 slides towards the stop 904, thereby moving the first lumen 114 away from any adjacent walls. While the single lumen insertion 902 is illustrated extending past the flex tip 112 in FIG. 10b, in other embodiments the single lumen insertion 902 may terminate within the flex tip 112 when in the neutral orientation.

FIG. 10c illustrates the centering assembly 110 in a collapsed orientation. In one example, when a force is applied on the proximal end of the centering assembly 110 in the direction of the distal end, typically during extraction, the centering assembly 110 moves along the distal extrusion until it flattens relative to the neutral orientation. This allows the centering assembly 110 to be easily removed by reducing the cross-sectional size of the centering assembly 110.

Another aspect of this disclosure includes a method of using the jet ventilation assembly 100. In one example, the jet ventilation assembly 100 may be packaged in a sterile pouch with a malleable stylet for insertion positioned within the conduit 102. The sterile pouch may further be delivered in a box. One or both of the sterile pouch and box may include printing thereon providing identifying information and/or instructions for use of the jet ventilation assembly 100, among other things. Further, in one aspect of this disclosure, the box may include an instruction pamphlet therein providing instructions for using the jet ventilation assembly 100 among other things.

One or more of the sterile pouch, the box, and/or the pamphlet may provide instructions for using the jet ventilation assembly 100. A user may reference the instructions, or otherwise be instructed to use the jet ventilation assembly 100 as follows. In box 802, the user may inspect that the box is intact and free for damage. The user may then open the box and remove the single sterile pouch containing the jet ventilation assembly 100 in box 804. In box 806, the user may inspect the sterile pouch and confirm that it is intact and free from damage, also confirm the expiration date to confirm product is valid. Next, the user may open the sterile pack towards the sterile field and allow staff to take hold of the proximal end of the jet ventilation assembly 100 by the luer fitting 104b and gently remove the jet ventilation assembly 100 from the sterile pack in box 808.

Once the patient is sedated and surgical team is ready, in box 810 the jet ventilation assembly 100 will be inserted and advanced to the desired position with the stylet in place. Once centering assembly 110 is in the subglottic region and the catheter is advanced to the correct position, the user may remove the stylet in box 812. In box 814, the user may connect 1^{st} Luer fitting 104b to a jet ventilator or other medical device to oxygenate and ventilate the patient. In box 816, the user may connect 2^{nd} Luer fitting 122b to a jet ventilator or other medical device to monitor tracheal pressure or gases. In box 818, the user may connect 3^{rd} Luer fitting 124b to a ventilator or other medical device to monitor end tidal CO2 (EtCO2), other gases or to monitor tracheal pressure. In box 820, when the procedure is done, the jet ventilation catheter can be removed, and normal operating room protocols followed and disposing the jet ventilation assembly 100 and stylet at termination of procedure.

In one aspect of this disclosure, the flex tip 112 is formed through an injection mold process, which allows the flex tip 112 to have a smooth and atraumatic outer surface that can easily progress along soft tissue of a patient without the risk of components separating from one another. Additionally, this process allows for a thru-hole of the flex tip 112 that allows the single lumen insertion 902 extend at least partially therethrough.

The process for forming the flex tip 112 and centering assembly 110 may be executed as follows. Among other things, this process involves forming the proximal end of the centering assembly 110 and joining it to the conduit 102. Traditional methods of heat bonding or welding cannot be formed between a PEBAX material and a PTFE material. Accordingly, the centering assembly 110 of the present disclosure is coupled to the conduit by heat welding all four flex flanges 130a, 130b, 130c, 130d together on the PTFE conduit 102 so as to create a smooth and near seamless transition between the conduit 102 and the proximal end of the centering assembly 110. Then, a primer and cyanoacrylate adhesive are applied to bond the centering assembly 110 to the conduit. Accordingly, the present disclosure involves forming the proximal end of the centering assembly 110 on the conduit 102 before gluing, which improves the transition, function, and appearance of the device.

While implementations incorporating the principles of the present disclosure have been described hereinabove, the present disclosure is not limited to the described implementations. Instead, this application is intended to cover any variations, uses, or adaptations of the disclosure using its general principles. Further, this application is intended to cover such departures from the present disclosure as come within known or customary practice in the art to which this disclosure pertains and which fall within the limits of the appended claims.

1. A jet ventilation assembly, comprising:
a conduit having a luer fitting on a proximal end and configured to fluidly couple the luer fitting to a distal end of the conduit; and
a centering assembly coupled to the conduit at the distal end;
wherein, the centering assembly has a flex tip that is movable along the conduit.

2. The jet ventilation assembly of claim 1, wherein the conduit defines a first lumen and a second lumen therein, the first lumen configured to provide oxygen to a patient and the second lumen configured to provide monitoring information to a user.

3. The jet ventilation assembly of claim 2, wherein the conduit defines a third lumen, the third lumen configured to provide additional monitoring information to a user.

4. The jet ventilation assembly of claim 2, wherein the first lumen terminates at a location proximate the flex tip and is configured to direct fluid through the first lumen out the distal end of the flex tip.

5. The jet ventilation assembly of claim 4, wherein the second lumen terminates at a monitoring zone of the centering assembly.

6. The jet ventilation assembly of claim 1, wherein the centering assembly comprises at least one flex flange fixedly coupled to the conduit on one end and coupled to the flex tip on the opposite end.

7. The jet ventilation assembly of claim 1, wherein the centering assembly comprises a plurality of flex flanges each fixedly coupled to the conduit on one end and coupled to the flex tip on the opposite end.

8. The jet ventilation assembly of claim 3, wherein the first lumen terminates at a location proximate the flex tip and the second and third lumen terminate at a monitoring zone of the centering assembly.

9. The jet ventilation assembly of claim 3, further comprising second luer fitting configured to fluidly couple the second lumen to a first medical device and a third luer fitting configured to couple the third lumen to a second medical device.

10. The jet ventilation assembly of claim 9, wherein the second lumen is configured to fluidly couple the first medical device to the monitoring zone to measure a pressure in a patent's trachea.

11. The jet ventilation assembly of claim 10, wherein the third lumen is configured to fluidly couple the second medical device to the monitoring zone to monitor CO2 levels.

12. The jet ventilation assembly of claim 1, wherein the conduit is formed of PTFE.

13. The jet ventilation assembly of claim 1, wherein the centering assembly is formed of PEBAX.

14. The jet ventilation assembly of claim 1, wherein the centering assembly is configured to elastically deflect to allow the flex tip to move along the conduit to absorb shock during insertion and removal.

15. The jet ventilation assembly of claim 1, wherein the conduit defines a stop configured to contact the flex tip when the centering assembly is moved to a maximum deflection.

16. A method of manufacturing a jet ventilation assembly, comprising:
injecting a material into a mold that defines a centering assembly having a flex tip and a plurality of flex flanges, wherein each of the flex flanges are formed extending from the flex tip;
forming a tri-lumen conduit through an extrusion process; and
coupling the centering assembly to the tri-lumen conduit by heat-welding the plurality of flex flanges to one another around the tri-lumen conduit.

17. The method of claim 16, wherein the flex tip is formed with a through hole and at least one lumen of the tri-lumen conduit is positioned at least partially in the through hole.

18. The method of claim 16, further comprising applying a primer and an adhesive to the flex flanges and the tri-lumen conduit to further couple the centering assembly to the tri-lumen conduit.

19. The method of claim 18, wherein the adhesive is a cyanoacrylate.

20. The method of claim 16, wherein the centering assembly is formed of PEBAX and the tri-lumen conduit is formed of PTFE.
